(19) United States
(12) Patent Application Publication
(10) Pub. No.: US 2023/0046804 A1
(43) Pub. Date: Feb. 16, 2023

(54) PAINLESS SYRINGE TO REDUCE THE NOISE AND FRICTION GENERATED WHEN PERFORATING THE ALVEOLAR BONE

(71) Applicant: Dentis Co., Ltd., Daegu (KR)

(72) Inventors: Gi Bong SIM, Daegu (KR); Jeong Hee SEO, Daegu (KR); Shin Wook PARK, Daegu (KR); Min Su KIM, Daegu (KR); Sun Min LEE, Daegu (KR)

(21) Appl. No.: 17/471,103

(22) Filed: Sep. 9, 2021

(30) Foreign Application Priority Data
Aug. 10, 2021 (KR) 10-2021-0104995

(51) Int. Cl. A61C 5/40; A61M 5/24

(52) U.S. Cl. CPC A61C 5/40; A61M 5/2425; A61M 2005/2485; A61M 2205/42; A61M 2205/0222; A61M 2210/02; A61M 2202/048; A61M 2005/2407

(57) ABSTRACT

According to an embodiment, a painless syringe for reducing noise and friction caused when perforating an alveolar bone comprises an injecting part having a needle for perforating the alveolar bone and injecting an anesthetic, a holder having a first end coupled with the injecting part and a second end open and empty, an ampoule formed to be inserted into the holder and having a first end into which the needle of the injecting part is inserted and a second end having a piston for discharging the anesthetic, a housing formed to receive the holder and having a through hole to expose the first end of the holder to an outside, and a handpiece coupled with the housing, rotating the ampoule, the holder, and the injecting part to perforate the alveolar bone, and pressing the piston to inject the anesthetic.

### CROSS-REFERENCE TO RELATED APPLICATION(S)

This application is based on and claims priority under 35 U.S.C. 119 to Korean Patent Application No. 10-2021-0104995, filed on Aug. 10, 2021, in the Korean Intellectual Property Office, the disclosure of which is herein incorporated by reference in its entirety.

### STATEMENT REGARDING SPONSORED RESEARCH OR DEVELOPMENT

This invention was made with Korean government support under Project No. S2782714 sponsored by the Ministry of SMEs and Startups (MSS) and managed by Korea Technology and information Promotion Agency for SMEs (TIPA) (under Research Project Title: Technical Development for Commercialization by SMEs(Development of new products subject to purchase conditions); Subject Title: Development of Auto-injection device with wireless charging for intra-osseous anesthetic in Oral; Research period: Oct. 1, 2019 through Sep. 30, 2019) with the primary beneficiary of sponsorship being Dentis Co., Ltd.

### TECHNICAL FIELD

The disclosure relates to a painless syringe for reducing noise and friction generated when perforating the alveolar bone, and more particularly, to a painless syringe for reducing the noise and friction generated when perforating the alveolar bone so as to prevent a reduction in rotational force due to friction when perforating the alveolar bone by rotating the needle upon injecting an anesthetic for dental treatment.

### DESCRIPTION OF RELATED ART

In general, local anesthesia means to inject anesthetic directly into a surgical site to anesthetize only the surgical site and is mainly used in dental implant surgery.

Conventionally, the operator directly fills the syringe with the anesthetic and then injects the anesthetic. If the injection rate of the anesthetic is increased, excessive pressure is generated in the affected area and the patient feels pain.

In other words, the pressure and rate of injecting anesthetic may be major factors that may cause pain during local anesthesia. Further, when the operator injects, the injection rate and pressure are not constant and the injection state cannot be numerically identified. Thus, the patient cannot but endure the pain caused during injection.

To address the issues, Korean Patent Application Publication No. 10-2019-0101062 discloses a painless anesthetic injection device capable of minimizing pain by determining an injection amount and injection rate when injecting an anesthetic into an affected area in the accurate location, which includes an ampoule part having one side connected to a connection part of a housing and the other side coupled to a needle part and receiving a solution inside, a plunger disposed inside the housing and applying pressure to the ampoule part to inject the solution through the needle part, a motor to move the plunger, and a controller to transmit a driving control signal to the motor according to an injection mode.

Use of such conventional art allows for a constant injection rate and control of the injection amount, thereby reducing the pain suffered by the patient. However, the conventional art does not address the issue that the operator is required to manually insert the needle up to the position of injection to inject the anesthetic to the inside the alveolar bone.

### SUMMARY

To address the foregoing issues, the disclosure aims to provide a painless syringe for reducing noise and friction caused when perforating the alveolar bone, which renders it possible to directly inject an anesthetic into the root canal by perforating the alveolar bone while rotating the needle upon injecting the anesthetic.

The disclosure also aims to provide a painless syringe for reducing noise and friction caused when perforating the alveolar bone, which renders it possible to stably perforate the alveolar bone by preventing a reduction in rotational force due to friction in the contact area of the portion of rotation of the needle.

The disclosure also aims to provide a painless syringe for reducing noise and friction caused when perforating the alveolar bone, which is allowed for long-term use by reducing wear and noise from the portion of rotation.

According to an embodiment, a painless syringe for reducing noise and friction caused when perforating an alveolar bone comprises an injecting part having a needle for perforating the alveolar bone and injecting an anesthetic, a holder having a first end coupled with the injecting part and a second end open and empty, an ampoule formed to be inserted into the holder and having a first end into which the needle of the injecting part is inserted and a second end having a piston for discharging the anesthetic, a housing formed to receive the holder and having a through hole to expose the first end of the holder to an outside, and a handpiece coupled with the housing, rotating the ampoule, the holder, and the injecting part to perforate the alveolar bone, and pressing the piston to inject the anesthetic.

An inner diameter of the housing may be larger than an outer diameter of the holder to prevent the holder from contacting an inner surface of the housing. The housing may contact an outer surface of the first end of the holder to support the holder.

The painless syringe may further comprise a bushing formed at a first end of the housing and contacting a supporting end formed at the first end of the holder to reduce friction caused when the holder rotates.

A contacting portion of the bushing may be rounded to reduce a contact area.

The bushing may have a lubrication groove formed in an inner surface thereof to receive or discharge a lubricant to reduce friction.

The bushing may be formed of a plastic bearing to reduce weight and noise due to friction.

According to the embodiments of the disclosure, the painless syringe for reducing noise and friction caused when perforating the alveolar bone renders it possible to directly inject an anesthetic into the root canal by perforating the alveolar bone while rotating the needle upon injecting the anesthetic.

According to the embodiments of the disclosure, the painless syringe for reducing noise and friction caused when perforating the alveolar bone renders it possible to stably perforate the alveolar bone by preventing a reduction in rotational force due to friction in the contact area of the portion of rotation of the needle.

According to the embodiments of the disclosure, the painless syringe for reducing noise and friction caused when perforating the alveolar bone is allowed for long-term use by reducing wear and noise from the portion of rotation.

### BRIEF DESCRIPTION OF THE DRAWINGS

A more complete appreciation of the disclosure and many of the attendant aspects thereof will be readily obtained as the same becomes better understood by reference to the following detailed description when considered in connection with the accompanying drawings, wherein:

FIG. 1 is a cross-sectional view illustrating a structure of an assembled painless syringe for reducing noise and friction caused when perforating the alveolar bone, according to an embodiment;

FIG. 2 is an enlarged view illustrating an injecting part of a painless syringe for reducing noise and friction caused when perforating the alveolar bone, according to an embodiment; and

FIG. 3 is a cross-sectional view illustrating a bushing for reducing noise and friction in a painless syringe for reducing noise and friction caused when perforating the alveolar bone, according to an embodiment.

### DETAILED DESCRIPTION

Specific features and advantages of the disclosure are described below in detail with reference to the accompanying drawings. When determined to make the gist of the disclosure unnecessarily unclear, a detailed description of the functions and features of the disclosure are omitted.

The disclosure relates to a painless syringe for reducing noise and friction generated when perforating the alveolar bone, and more particularly, to a painless syringe for reducing the noise and friction generated when perforating the alveolar bone so as to prevent a reduction in rotational force due to friction when perforating the alveolar bone by rotating the needle upon injecting an anesthetic for dental treatment.

Hereinafter, exemplary embodiments of the disclosure are described in detail with reference to the accompanying drawings.

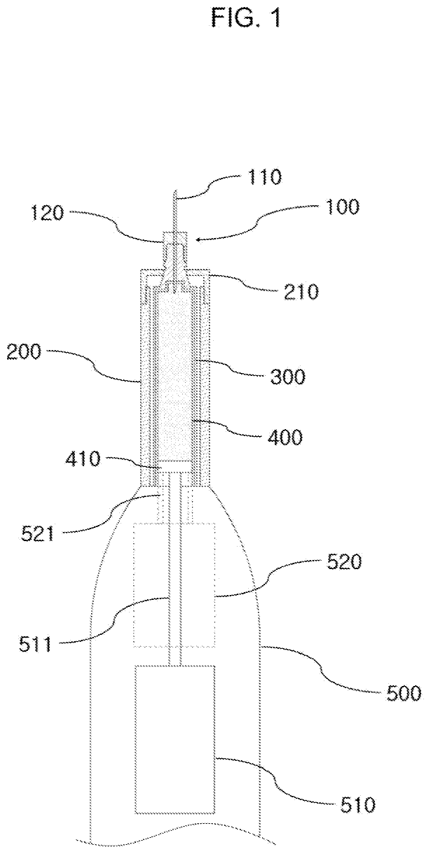

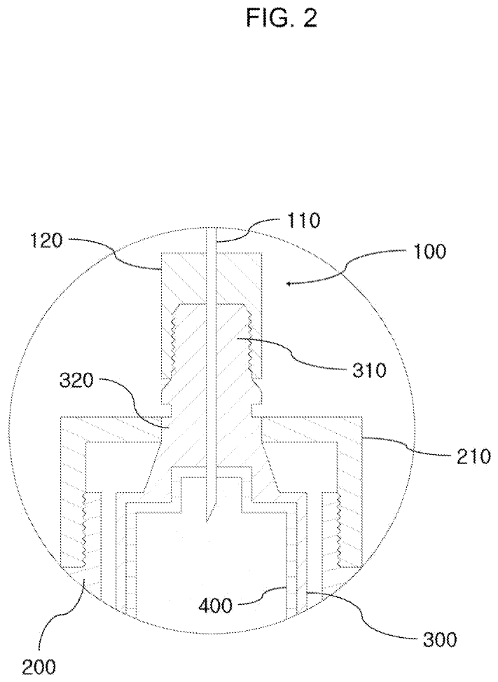

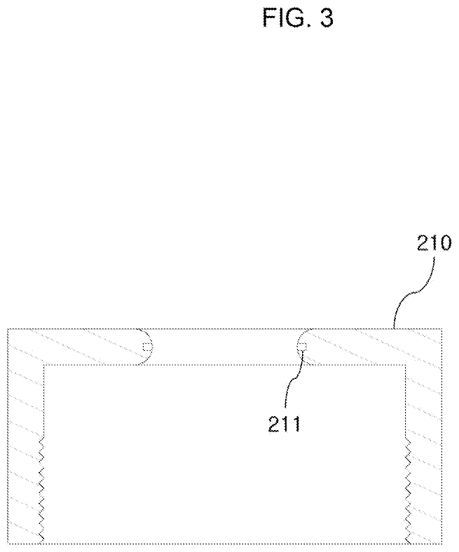

FIG. 1 is a cross-sectional view illustrating a structure of an assembled painless syringe for reducing noise and friction caused when perforating the alveolar bone, according to an embodiment. FIG. 2 is an enlarged view illustrating an injecting part 100 of a painless syringe for reducing noise and friction caused when perforating the alveolar bone, according to an embodiment. FIG. 3 is a cross-sectional view illustrating a bushing for reducing noise and friction in a painless syringe for reducing noise and friction caused when perforating the alveolar bone, according to an embodiment.

Referring to FIGS. 1 to 3, according to an embodiment, a painless syringe for reducing noise and friction caused when perforating an alveolar bone includes an injecting part 100 having a needle 110 for perforating the alveolar bone and injecting an anesthetic, a holder 300 having a first end coupled with the injecting part and a second end open and empty, an ampoule 400 formed to be inserted into the holder 300 and having a first end into which the needle 110 of the injecting part is inserted and a second end having a piston 410 for discharging the anesthetic, a housing 200 formed to receive the holder 300 and having a through hole to expose the first end of the holder 300 to an outside, and a handpiece 500 coupled with the housing 200, rotating the ampoule 400, the holder 300, and the injecting part 100 to perforate the alveolar bone, and pressing the piston 410 to inject the anesthetic.

The injecting part 100 includes a support 120 and the needle 110. The support 120 is formed in a cylindrical shape and has a first surface open to allow the first end of the holder 300 to be inserted thereto. The needle 110 is formed in the center of the support 120 and passes through the first surface and a second surface of the support 120 to discharge the anesthetic received inside the ampoule 400 or to perforate the alveolar bone.

The handpiece 500 includes a first driver 510, a second driver 520, a first driving end 511, and a second driving end 521. The first driver 510 and the second driver 520 generate rotational force by external power. The first driving end 511 is coupled with the piston 410 and converts the rotational force by the first driver 510 into translational motion to move back and forth the piston 410. The second driving end 521 is coupled with the ampoule and rotate the ampoule by the second driver 520, thereby rotating the holder 300 and the injecting part 100 coupled with the ampoule 400.

The support 120 of the injecting part 100 is detachably attached to the first end of the holder 300 to replace the needle 110 and is screwed to the outer surface of the first end of the holder 300. The needle 110 protrudes to two opposite sides of a center of the support 120, so that a first end of the needle 110 perforates the alveolar bone to inject the anesthetic into the root canal, and a second end of the needle 110 is inserted through the holder 300 to the inside of the ampoule to discharge the anesthetic stored in the ampoule 400.

Since the support 120 and the needle 110 are integrally formed, if the injection is completed or the procedure is finished, the injecting part 100 may be removed and discarded, so that a medical accident may be prevented.

The first and second ends of the needle 110 are formed with sharp tips (or bevels) so that the first end of the needle 110 is inserted, with reduced resistance, into the patient's gums and alveolar bone, and the second end of the needle 110 may pierce the ampoule 400 into the inside of the ampoule 400 by its sharp tip (or bevel).

The first end of the holder 300 is formed to allow the injecting part 100 to be detachably attached thereto, and the second end of the holder 300 is open to allow the ampoule to be inserted thereinto. The injecting part 100 may be fixed to the first end of the holder 300, and the ampoule may be fixed to the second end of the holder 300.

The first end of the holder 300 is provided with a fastening end 310 threated to be coupled with the injecting part 100, and a rotating end 320 is formed on the rear surface of the fastening end 310 to be fixed by the housing 200.

The fastening end 310 is formed to be screwed with the support 120 of the injecting part 100 to be detachably attached to the support 120, and the rotating end 320 is formed to contact the housing 200 to allow the first end of the holder 300 to be fixed to the housing 200.

The rotating end 320 is rotated in contact with the housing 200 when the holder 300 is rotated by the driver of the handpiece 500, and a lubricant (e.g., a lubricating oil) may be applied to the outer surface of the rotating end 320 to reduce friction.

The ampoule 400 may be formed in a shape corresponding to (or identical to) the inner shape of the holder 300 to tightly contact the inner surface of the holder 300, and the first end of the ampoule 400 may be formed of rubber to allow for insertion of the needle 110 of the injecting part 100 coupled to the first end of the holder 300.

The second end of the ampoule 400 is open to receive and store an anesthetic therein and is coupled with the piston 410 and sealed to prevent leakage of the stored anesthetic.

The housing 200 is formed at a first end of the handpiece 500 and is detachably attached to the handpiece 500. The inside of the housing 200 is formed to allow the holder coupled with the injecting part 100 and the ampoule 400 to be attached or detached.

The first end of the housing 200 has a through hole through which the first end of the holder 300 protrudes to the outside of the housing 200. The injecting part 100 coupled to the first end of the holder 300 may be exposed to the outside through the through hole.

The handpiece 500 may be used to control the operation of automatic injection and to receive related devices and may include a controller, a battery, a display, a manipulation unit, a speaker, a charging unit, and a pressure sensor.

Drivers of the handpiece 500 may be divided into a first driver 510 and a second driver 520 which include a first driving end 511 and a second driving end 521, respectively, to perform different operations.

The first driver 510 and the second driver 520 receive power from the battery and generate rotational force.

The first driving end 511 may be formed to be coupled with the piston 410 formed inside the ampoule 400. Upon receiving the rotational force from the first driver 510, the first driving end 511 may convert the rotational force into translational motion and be moved up or down to thereby move the piston 410.

The second driving end 521 is coupled with the second end of the ampoule 400 and, if receiving rotational force from the second driver 520, transfers the rotational force to the ampoule 400 to rotating the ampoule 400, thereby rotating the holder 300 and injecting part 100 coupled with the ampoule 400 and thus allowing the needle 110 of the injecting part 100 to perforate the alveolar bone.

In this case, the second driving end 521 may be coupled to the holder 300, not the ampoule 400, to rotate the holder 300.

The second driving end 521 may be hollow to position the first driving end 521 in the center thereof.

If the second end of the housing 200 is coupled to the handpiece 500, the ampoule 400 positioned inside the housing 200 is positioned coaxial with the first driving end 511 formed inside the handpiece 500 and, if the first driving end 511 is moved by the first driver 510 of the handpiece 500 to be coupled to the rear surface of the piston 410.

It is preferable that the second driving end 521 is also formed so that when the housing 200 is coupled to the handpiece 500, the second driving end 521 is coupled with the ampoule 400 or the holder 300 in substantially the same manner as the first driving end 511.

The inner diameter of the housing 200 is larger than the outer diameter of the holder 300 to prevent the holder 300 from contacting the inner surface of the housing 200 to reduce the contact area when the holder 300 is inserted in the housing 200. The housing 200 contacts the outer surface of the first end of the holder 300 to support the holder 300.

The inner surface of the housing 200 does not contact the outer surface of the holder 300 but is spaced apart from the outer surface of the holder 300. However, the first end of the holder 300 is inserted through the through hole formed in the first end of the housing 200 and contacts the first end of the housing 200.

In this case, the rotating end 320 of the holder 300 is inserted through the through hole of the housing 200 and contacts the housing 200. The second end of the holder 300 is supported by the first driving end 511 and the second driving end 521 of the handpiece 500 to be fixed in position.

To keep the housing 200 spaced apart from the holder 300, the inner diameter of the housing 200 is larger than the outer diameter of the holder 300. Thus, when the holder 300 is rotated, interference or friction may be prevented by the gap.

A bushing 210 is formed at the first end of the housing 200. The bushing 210 contacts a supporting end formed at the first end of the holder 3001 to reduce friction caused when the holder 300 rotates.

The through hole-formed portion of the first end of the housing 200 may be replaced with the bushing 210.

In this case, the bushing 210 may have a first surface with a through hole through which the rotating end 320 of the holder 300 may be inserted and a second surface formed to be screwed with the outer surface of the housing 200.

The bushing 210 is detachable from the housing 200. Thus, if the portion of the bushing 210 in contact with the rotating end 320 is worn by the rotation of the holder 300, the worn bushing 210 may be removed from the housing 200, and a new bushing 210 may be coupled. Thus, simplified maintenance may be achieved.

To reduce the contact area, the contacting portion of the bushing 210 may be rounded in a circular shape as shown in FIG. 3.

A lubrication groove 211 may be formed in the inner contacting surface of the bushing 210 to receive or discharge a lubricant so as to reduce friction, as shown in FIG. 3.

The bushing 210 may be formed of a plastic bearing to reduce weight and reduce noise generated by friction.

The bushing 210 may be formed of a plastic material to prevent abrasion of the contacting holder 300 and to reduce weight. The bushing 210 may serve as a bearing to stably support the rotating holder 300 and prevent a decrease in the rotational speed of the holder 300 due to friction at the contacting portion.

Heat or noise caused by friction may be reduced by decreasing friction through the bushing 210.

In particular, the portion contacting the rotating end 320 of the holder 300 is rounded to minimize the contact area, and the lubrication groove 211 is formed in the central inner surface of the contacting portion to receive a lubricant.

As a lubricant is received in the lubrication groove 211, the lubricant may be applied to the portion contacting the rotating end 320, reducing friction at the contacting portion.

The lubrication groove 211 is shaped as a capillary tube, so that the lubricant may be prevented from flowing down by gravity but rather remain stored in the lubrication groove 211. As the lubricant contacts the rotating end 320, the lubricant may come out to the surface of the rotating end 320 by surface tension, maintaining lubricity.

If the lubricant is dried on the surface of the rotating end 320, the lubricant contained in the lubrication groove 211 may come into contact with the rotating end 320 and partially flow out due to surface tension, thereby allowing an appropriate amount of lubricant to be applied to the rotating end 320.

As described above, in the painless syringe for reducing noise and friction caused when perforating the alveolar bone, the needle may be rotated to perforate the alveolar bone and directly inject the anesthetic to the root canal. A decrease in rotational force due to friction in the contact area of the rotating portion when the needle is rotated may be prevented, leading to stable perforation. Further, it is possible to reduce noise from the rotating portion and abrasion, allowing for long-term use.

Although preferred embodiments of the disclosure have been described above, various changes or modifications may be made thereto by one of ordinary skill in the art without departing from the technical spirit and scope of the claims. Accordingly, the scope of the disclosure should be interpreted by the following claims described to include such various changes.

What is claimed is:

1. A painless syringe for reducing noise and friction caused when perforating an alveolar bone, comprising:
an injecting part having a needle for perforating the alveolar bone and injecting an anesthetic;
a holder having a first end coupled with the injecting part and a second end open and empty;
an ampoule formed to be inserted into the holder and having a first end into which the needle of the injecting part is inserted and a second end having a piston for discharging the anesthetic;
a housing formed to receive the holder and having a through hole to expose the first end of the holder to an outside; and
a handpiece coupled with the housing, rotating the ampoule, the holder, and the injecting part to perforate the alveolar bone, and pressing the piston to inject the anesthetic.

2. The painless syringe of claim 1, wherein an inner diameter of the housing is larger than an outer diameter of the holder to prevent the holder from contacting an inner surface of the housing, and wherein the housing contacts an outer surface of the first end of the holder to support the holder.

3. The painless syringe of claim 1, further comprising a bushing formed at a first end of the housing and contacting a supporting end formed at the first end of the holder to reduce friction caused when the holder rotates.

4. The painless syringe of claim 3, wherein a contacting portion of the bushing is rounded to reduce a contact area.

5. The painless syringe of claim 3, wherein the bushing has a lubrication groove formed in an inner surface thereof to receive or discharge a lubricant to reduce friction.

6. The painless syringe of claim 3, wherein the bushing is formed of a plastic bearing to reduce weight and noise due to friction.
